(19) United States
(12) Patent Application Publication
(10) Pub. No.: US 2003/0026758 A1
(43) Pub. Date: Feb. 6, 2003

(54) Method and device for monitoring real-time position of an area targeted by a radiosurgery system

(76) Inventor: Gregg S. Baker, Geneva, FL (US)

Correspondence Address: Finnegan, Henderson, Farabow,, Garrett & Dunner, L.L.P., 1300 I Street, N.W., Washington, DC, 20005-3315, US

(21) Appl. No.: 10/205,486

(22) Filed: Jul. 26, 2002

Related U.S. Application Data
(60) Provisional application No. 60/307,856, filed on Jul. 27, 2001.

(51) Int. Cl. A61K 49/00; G06F 19/00; G01N 33/48; G01N 33/50; G06K 9/00; A61B 5/00

(52) U.S. Cl. 424/9.1; 600/300; 702/19; 382/128

(57) ABSTRACT

A system and process for monitoring a targeted area of a patient's body to precisely locate the targeted area in real-time are provided. The system includes at least one datum device to be placed a predetemined distance from the targeted area, either on or within the body. The system also includes means for sensing the at least one datum device, means for determining a real-time position of the at least one datum device, and means for calculating changes in the position of the datum device in real-time. Any changes in the position of the datum device are conveyed to a radiotherapy device which emits a radiation beam directed at the targeted area. The radiation beam is controlled according to changes in the position of the datum device in order to maintain precise targeting on the area to be treated.

This application claims priority under 35 U.S.C. § 119 based on U.S. Provisional Application No. 60/307,856, filed Jul. 27, 2001, the complete disclosure of which is incorporated herein by reference.

### DESCRIPTION OF THE INVENTION

1. Field of the Invention

The present invention relates to a method and system for precisely targeting a tumor to be treated and for tracking changes in the relative position of the tumor such that the position of the tumor can be accurately pinpointed in real-time.

2. Background of the Invention

Historically, patients having tumors are subjected to whole or localized radiation therapy to treat the tumors. Because a tumor may be located near healthy, sensitive tissue, a whole treatment is generally given and may last for four to five weeks. Such a whole treatment must be administered at a low dose in order to prevent serious injury to the non-diseased areas of the body.

Radiosurgery allows a highly concentrated beam of radiation to be focused on the tumor itself. Because the tumor can be targeted, the time for the radiation therapy can be greatly reduced. However, because the dose of the radiation used is extremely high, it is necessary to accurately identify the location of the tumor to be targeted. Radiation delivered improperly may cause significant damage to the nearby healthy tissue. If a tumor is located in such a manner that it is not possible to identify its position with a high level of confidence, radiosurgery is not indicated as the preferred method of treatment, especially if the tumor is located near critical tissue such as the nervous system. Radiosurgery is also a less preferred method of treatment when the tumor is located such that its position changes, for example, the tumor moves as the patient breathes, making it difficult to determine the position of the tumor in a real-time, dynamic mode.

Thus, there is a need that allows for identification of a location of a tumor in a real-time dynamic mode to permit for tracking of changes in the relative position of the tumor due to, for example, respiratory motion. There is also a need for a reliable, non-invasive manner of accurately identifying the position of a tumor to be treated when it is not located near bone or other dense, non-moving tissue.

### SUMMARY OF THE INVENTION

In accordance with the invention, a system and process for tracking changes in the position of a tumor to be treated in a real-time, dynamic mode is provided. The method and apparatus track changes of the position of a datum device relative to the targeted area that includes the tumor, precisely targeting the position of the datum device and thereby facilitating determination of the position of the tumor at any given point in time, regardless of movement of the datum device and tumor. Changes in the position of the datum device are conveyed to a radiotherapy device, which controls the output of the device accordingly.

According to one aspect of the invention, a method of tracking, in real-time, a position of a targeted area of a patient's body is provided. The method comprises placing at least one datum device a predetermined distance from the targeted area, substantially continuously sensing the at least one datum device, and repeatedly calculating the real-time position of the at least one datum device.

According to another aspect of the invention, a system for tracking, in real-time, a position of a targeted area of a patient's body is provided. The system comprises at least one datum device, means for determining the real-time position of the at least one datum device, means for calculating changes in the position of the at least one datum device, and means for conveying change in position information.

According to a further aspect of the invention, a method of surgically treating a tumor is provided. The method includes placing at least one datum device a predetermined distance from a targeted area, wherein the tumor is contained within the targeted area, sensing the at least one datum device, determining the position of the at least one datum device, conveying information about the position of the at least one datum device to a surgical device for treating the tumor, treating the tumor, repeatedly calculating changes in the position of the at least one datum device, conveying information about changes in the position of the at least one datum device to the surgical device, and controlling the surgical device in response to the conveyed information.

According to yet another aspect of the invention, a system for surgically treating a tumor is provided. The system includes at least one datum device, a surgical device for treating a tumor, means for determining the position of the at least one datum device, means for calculating changes in position of the at least one datum device in real-time, means for conveying change in position information to the surgical device, and means for controlling surgical device in response to the conveyed information.

According to another aspect of the invention, a method of surgically treating a tumor comprises applying a radiation beam to an area containing a tumor, tracking changes in the position of the area in real-time, and controlling the radiation beam in response to changes in the position of the area.

According to a further aspect of the invention, a system for surgically treating a tumor comprises means for applying a radiation beam to an area containing a tumor, means for tracking changes in the position of the area in real-time, and means for controlling the radiation beam in response to changes in the position of the area.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate one embodiment of the invention and together with the description, serve to explain the principles of the invention.

### BRIEF DESCRIPTION OF THE DRAWINGS

FIG. 1A is an axial view of a tissue section containing a tumor and a datum device, according to one aspect of the present invention;

FIG. 1B is an axial view of a tissue section containing a tumor and three datum devices, according to another aspect of the present invention;

FIG. 2 is a schematic diagram of a system according to the present invention;

FIG. 3 is a flow chart showing a process of tracking a datum device and controlling an automated surgical device with an adjustable output in response to changes in the position of the datum device according to the present invention;

FIG. 4 is a flow chart showing a process of tracking a datum device and controlling an automated surgical device with a fixed output in response to changes in the position of the datum device according the present invention; and

FIG. 5 is a flow chart showing a process of tracking a datum device and controlling a manually operated surgical device with a fixed output in response to changes in the position of the datum device according the present invention.

### DESCRIPTION OF THE EMBODIMENT

Reference will now be made in detail to the present embodiment of the invention, an example of which is illustrated in the accompanying drawings. Wherever possible, the same reference numbers will be used throughout the drawings to refer to the same or like parts.

According to one aspect of the present invention, a system for tracking, in real-time, the position of a targeted area of a patient's body is provided. The system 100 includes at least one datum device, means for sensing the position of the datum device, means for determining the position of the datum device, means for calculating a change in position of the datum device, means for conveying datum position information to a surgical treatment device, and means for controlling the output of the surgical treatment device in response to a change of position in the datum device and targeted area.

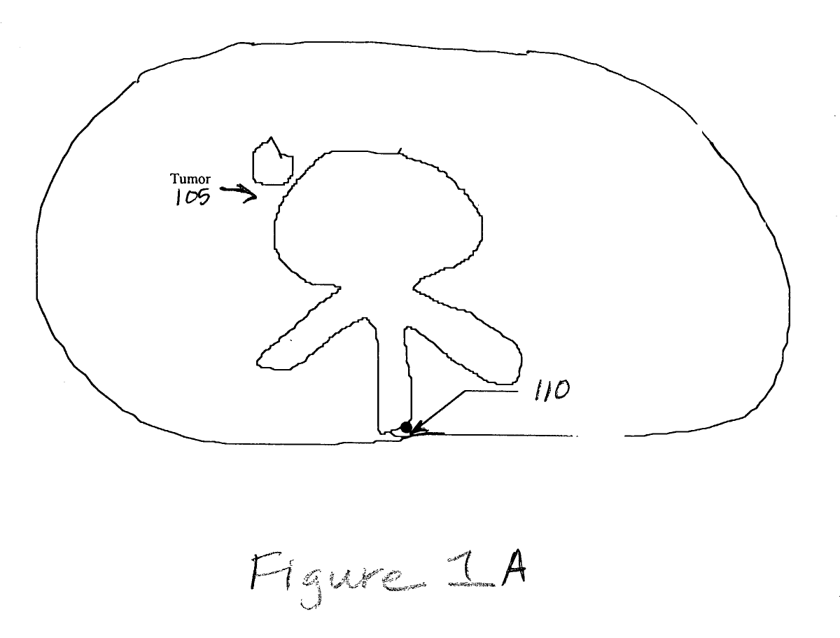

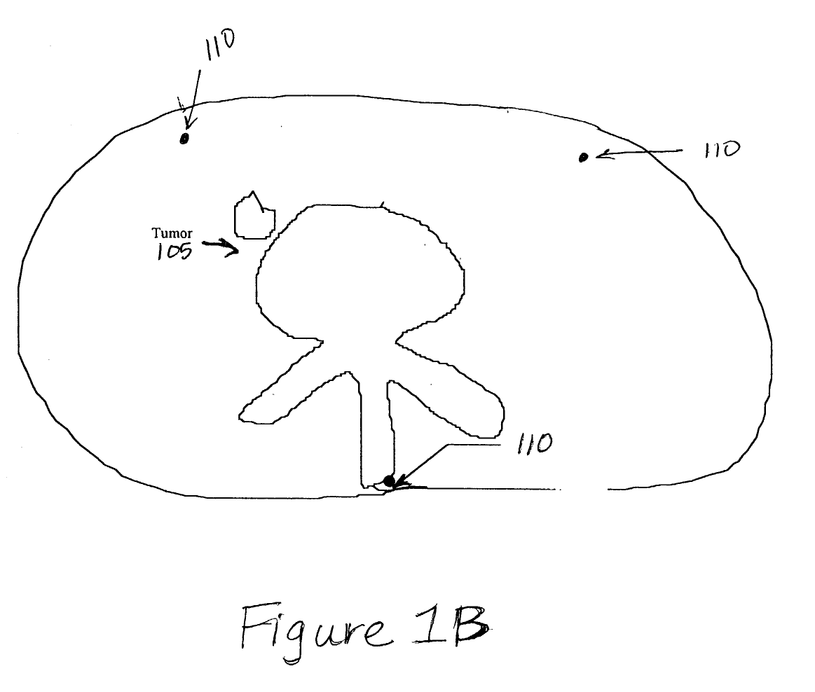

As embodied herein and shown in FIG. 1, at least one datum device 110 is provided. The purpose of the datum device 110 is to emit a signal or to affect a signal or condition, allowing its position to be tracked in real-time. By tracking the position of datum device 110, the position of any tissue, including any tumor, a predetemined distance from datum device 110 can also be tracked. It is not necessary that the datum device be positioned immediately adjacent the targeted area. As long as the distance between the datum device and the targeted area is known, the position of the targeted area may be monitored. Thus, the datum device is positioned a predetermined distance away from the targeted area, such that any change of position of the datum device can be used to determine a change in position of the targeted area.

Additionally, it is possible that more than one datum device 110 may be used to isolate a particular area of interest, and the position of the area may be determined by triangulation of the position of the datum devices 110. The number of datum devices 110 used would depend, for example, on the sensitivity and size of the area to be targeted. FIG. 1B shows three datum devices 110 in use. Datum device 110 may or may not be implantable. If datum device 110 is implantable, a surgeon will implant the device prior to radiotherapy. If datum device 110 is not implantable, it should be configured to be attached externally on or near the body of a patient to be treated.

According to one aspect of the invention, datum device 110 may be an accelerometer. Acceleration signals generated by the accelerometer may be transmitted via a wire connected to a means for determining the position and velocity of the datum device 110. Alternatively, datum device 110 may be a device which generates a signal, such as an electromagnetic signal, a radio signal, or radiation, which could be detected by a means for determining the position of the datum device 110. Other types of devices which generate or affect a suitable type of signal may also be used as a datum device.

Alternatively, the datum device 110 may not emit a signal. Instead, the datum device 110 may be a passive marker which can be sensed remotely. For example, the datum device 110 may include, for example, a titanium bead, and the position of the datum device 10 may be remotely sensed by, for example, an ultrasonic device or an inductive proximity sensor.

As embodied herein, the system 100 for tracking, in real-time, the position of a targeted area of a patient's body includes a means 120 for determining the position of the datum device 110. The type of means 120 used to determine the position of the datum device 110 depends upon the type of datum device 110 used. For example, if datum device 110 is an accelerometer, the means 120 for determining the position of the datum device 110 may include a signal receiver which has the capability to interpret the acceleration signal(s) and then, based on the signals, calculate the position and velocity of the datum device 110. Alternatively, if datum device 110 generates a radio signal, the means may include a simple receiver. In such an embodiment, the position of the datum device 110 is determined by the direction and amplitude of the signal or by using multiple receivers to triangulate position. In addition, because more than one datum device may be used, it is preferable that the means 120 for determining the position of the datum device 110 includes the capability to determine position of the datum device(s) 110 via triangulation. In another alternative, the datum device may not emit any signal, and instead a sensor, such as a proximity sensor, may be used.

The means 120 for determining the position of the datum device 110 substantially continuously receives a signal from the datum device 110 and repeatedly calculates and recalculates the position of the datum device based on the signal. As used herein, the term “substantially continuously” means that, for practical purposes, the datum device position is monitored continuously. However, the signal emitted by the datum device, or the sensor used to locate the datum device, may not emit a continuous signal. Instead, the signal may be pulsed at a particular frequency, for example, 100 or 1000 Hertz. Minute interruptions of the signal are intended to be encompassed within the term “substantially continuously.”

At any given time, the means 120 for determining the position of the datum device 110 may use the signal/sensing of the datum device at that instant to calculate the position of the datum device, and thus the position of the targeted area, in real-time. By substantially continuously monitoring the position of the datum device 110, any change in position of the datum device 110 and the targeted area due to movement of the patient's body is accounted for in the calculations. Means for calculating a change in position of the data device 110 (and targeted area) substantially continuously compare newly calculated positions of the datum device 110 with previously calculated position information. Changes in the position are calculated by system software and are then supplied to a radiotherapy device by a means 140 for conveying change in position information, for example, via coordinate output.

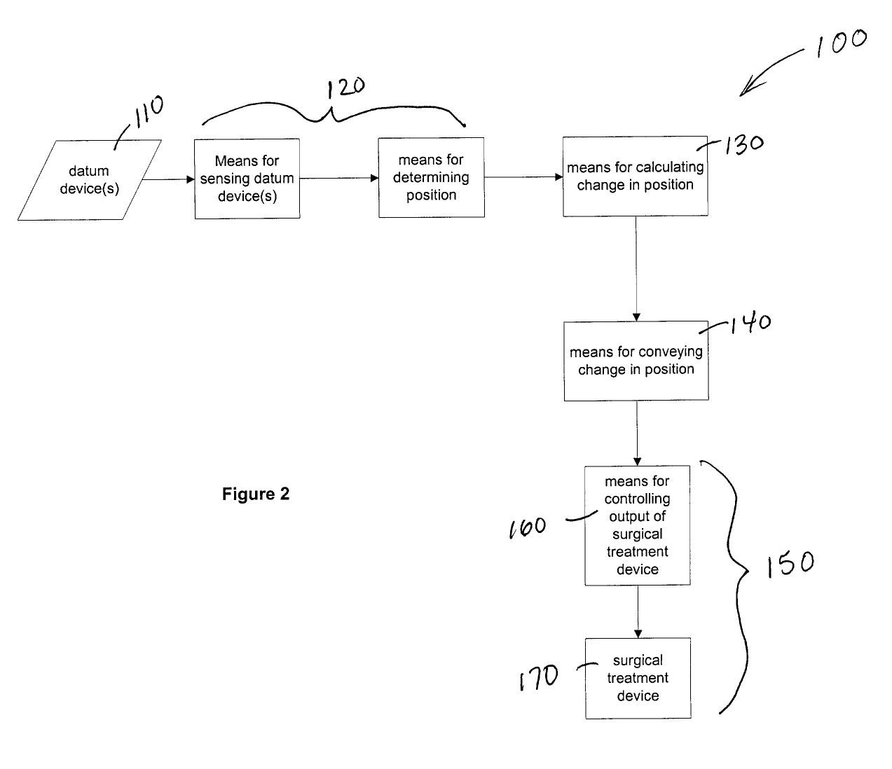

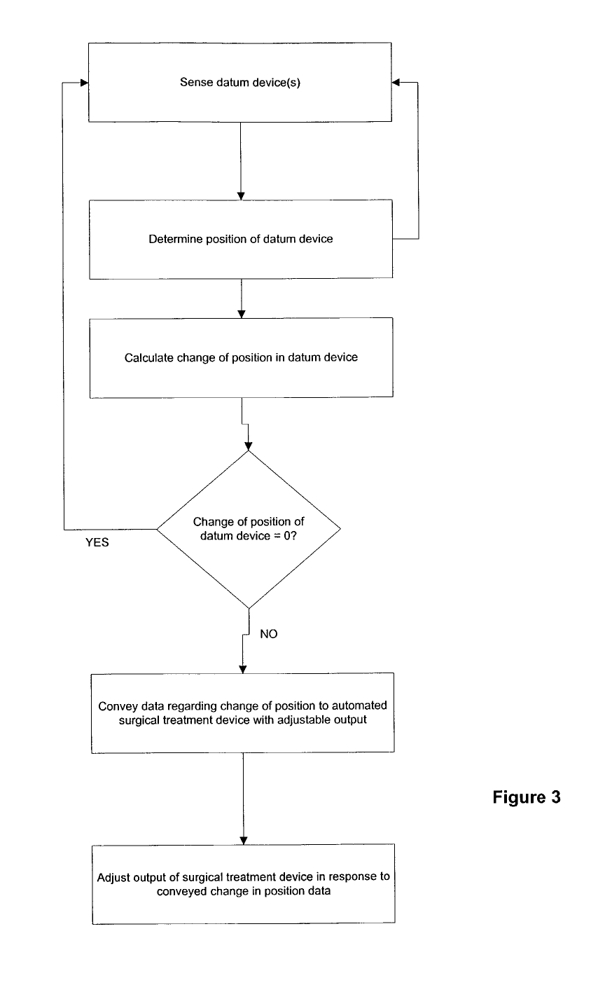

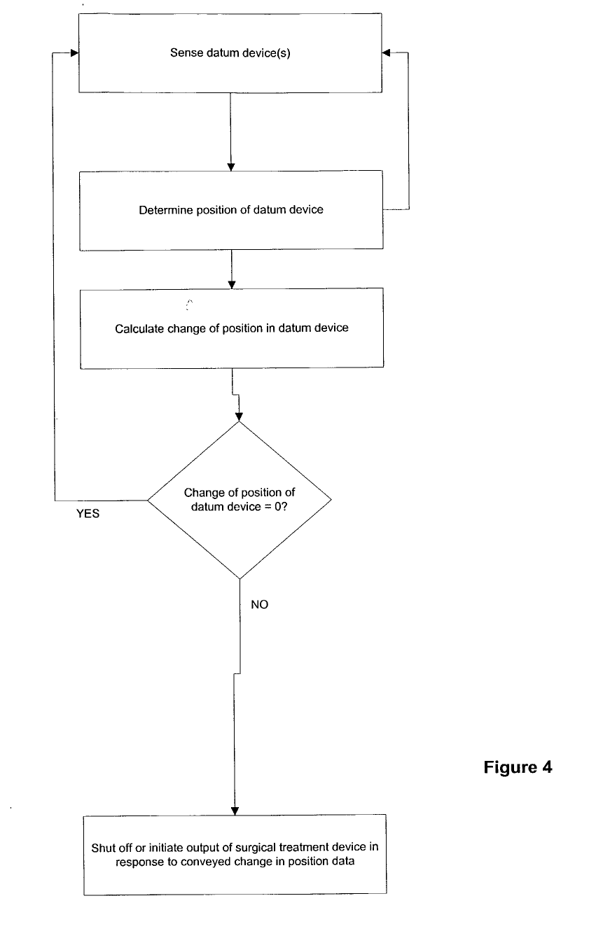

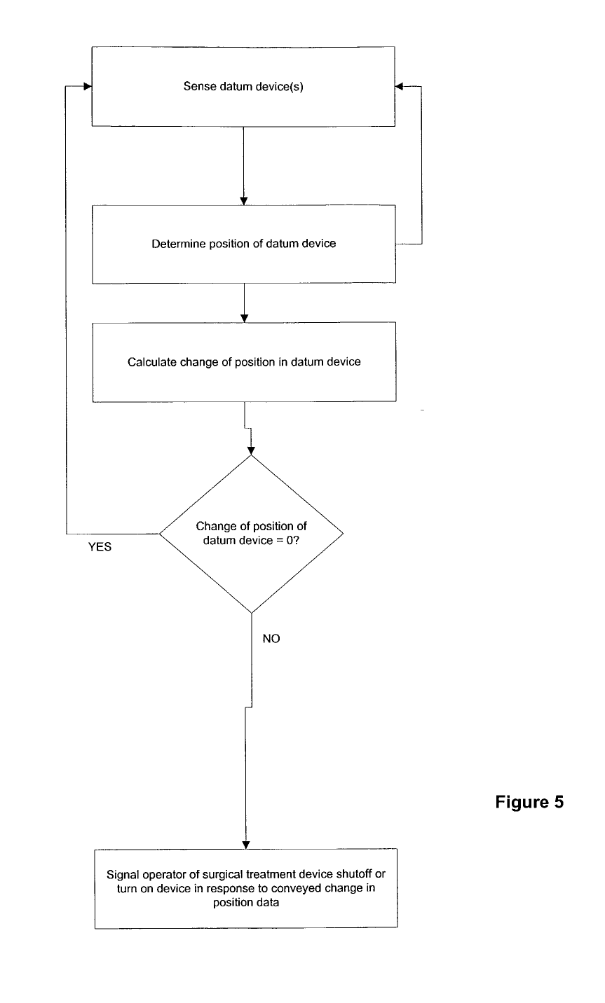

As embodied herein and shown in FIG. 2, the system 100 includes a surgical treatment device such as a radiotherapy device 150 which applies a radiation beam to a targeted site. Preferably, the radiotherapy device includes a linear accelerator 170 for emitting a beam of radiation of a desired intensity in a desired direction at a desired distance from the patient. Alternatively, other types of treatment devices with appropriate treatment capability may be used to treat the targeted area. The surgical treatment device may be automated and have an adjustable output (FIG. 3) or a fixed output (FIG. 4). Alternatively, the surgical device may be run manually by a technician (FIG. 5).

The radiotherapy device 150 also includes a receiver which receives information conveyed from the means for calculating change in position of the datum device 110 and means 160 for controlling the beam of radiation in response to the conveyed information. If information conveying a change in the datum device position is received, the beam of radiation emitted by the radiotherapy device is controlled accordingly. The means 160 for controlling the beam of radiation may adjust at least one of the angle, direction, intensity, or distance of the beam of radiation in response to the changed position of the datum device 110 and targeted area. Alternatively, the means 160 for controlling the beam of radiation may act as a switch, turning the beam on and off in response to the changed position of the datum device 110 and targeted area.

In use, as shown in FIGS. 1A and 3-5, a surgeon or technician implants or attaches at least one datum device 110 a predetemined distance from to an area to be treated on a patient, such as an area having a tumor or growth 105. The exact location for placement of the datum device(s) 110 may be determined from x-rays or other mapping of the patient's body. After placement of the at least one datum device 110, the patient is placed in a radiotherapy treatment theatre. Scans are taken to determine the position of the at least one datum device 110. Another scan is taken to establish the position of the datum device 110 relative to the area to be treated. From this information, it is possible to determine the tumor's position relative to the datum device 110. Based upon the calculated position of the datum device 110 and area to be treated, radiosurgery on the area to be treated begins.

The means 120 for determining the position of the datum device substantially continuously receives a signal from the datum device 110 and repeatedly recalculates the position of the datum device 110 based on the signal. The means for calculating change in position repeatedly compares the real-time position of the datum device 110 with the previously calculated position. If the position of the datum device 110 changes, this information is conveyed to the radiotherapy device 150, and the output of the device is controlled, for example, by turning the radiation beam on or off, or by adjusting some aspect of the beam such as its direction, intensity, or angle. If the surgical device is not automated, the operator of the surgical device is instructed, for example by a flashing light, to turn the device on or off. This process continues throughout the surgery.

Although this process has been discussed with respect to the treatment of tumors, it is also possible that the system 100 of the present invention could be used for the treatment of any soft tissue area in which it would be difficult to monitor position of the area to be treated due to a lack of reference points. For example, such a system may be used in the treatment of organs such as the kidney, lungs, or liver. In addition, this process has been discussed with respect to the use of radiosurgery, and specifically, treatment with a radiation beam for therapy. It is possible, however, that the general process described, that of monitoring in real-time the position of a targeted area of a patient's body and conveying real-time changes in position of the targeted area to a computer-controlled surgical instrument, may be used in other surgical processes such as taking a biopsy of a tissue or otherwise controlling a surgical instrument.

Alternatively, it is envisioned that the surgical instrument may not be computer controlled, but rather operated by a surgeon. In such a case, the datum device 110 may cooperate with the surgical device to guide the surgeon, for example, by emitting a first, “good” sound or other suitable signal when the surgeon is close to the targeted area and a second “bad” sound or other suitable signal if the surgeon is more than a predetermined acceptable distance away from the targeted area.

According to another aspect of the present invention, it is contemplated that the above described surgical methods could be practiced remotely, for example, via the internet. In such an embodiment, a local team/doctor would place/implant the datum device(s) 110 a predetemined distance from the area to be treated. The datum device(s) 110 would be connected to a CPU which would convey signals emitted by the datum device(s) 110 to a central location. The central location would be staffed by a fully trained surgical team. The signals emitted by the datum device(s) 110 would be monitored and changes in position calculated as described above. Based upon these calculated real-time changes in position, one of the fully trained surgical team would determine the necessary changes to be made in angle, intensity, distance, of the beam and would provide direction, via the internet or remote control, to the linear accelerator to proceed with radiotherapy on the targeted site.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of tracking, in real-time, a position of a targeted area of a patient's body, comprising:
placing at least one datum device a predetemined distance from the targeted area;
substantially continuously sensing the at least one datum device; and
repeatedly calculating the real-time position of the at least one datum device.

2. The method of claim 2, further comprising:
determining if the position of the at least one datum device has changed; and
when the position of the datum device has changed, conveying information about the change of position to a computer-controlled surgical instrument.

3. The method of claim 3, further comprising:
controlling the computer-controlled surgical instrument in response to the conveyed change of position information.

4. The method of claim 3, wherein controlling the computer-controlled surgical instrument includes adjusting the output of surgical instrument.

5. The method of claim 4, wherein the output of the surgical instrument is a beam of radiation, and wherein adjusting the output of the surgical instrument includes adjusting at least one of an angle, a direction, an intensity, and a distance of the beam of radiation.

6. The method of claim 3, wherein controlling the surgical instrument includes turning the instrument on and shutting the instrument off.

7. The method of claim 1, wherein sensing the at least one datum device includes receiving a signal emitted by the at least one datum device.

8. The method of claim 1, wherein sensing the at least one datum device includes using a proximity sensor to locate the at least one datum device.

9. A system for tracking, in real-time, a position of a targeted area of a patient's body, comprising:
at least one datum device;
means for determining the real-time position of the at least one datum device;
means for calculating changes in the position of the at least one datum device; and
means for conveying change in position information.

10. The system of claim 9, further comprising a scanner.

11. The system of claim 9, wherein the means for conveying conveys information to a computer controlled surgical instrument.

12. The system of claim 9, wherein the datum device is an accelerometer.

13. The system of claim 9, wherein the datum device is configured to emit a signal.

14. The system of claim 9, wherein the datum device is implantable.

15. The system of claim 9, further comprising means for receiving a signal from the at least one datum device.

16. The system of claim 9, wherein the means for determining the real-time position of the at least one datum device includes a proximity sensor for sensing the at least one datum device.

17. The system of claim 9, further comprising means for controlling a computer controlled surgical instrument in response to conveyed change in position information.

18. The system of claim 17, wherein the surgical instrument is a radiotherapy device that emits a beam of radiation.

19. The system of claim 18, wherein the means for controlling the computer controlled surgical instrument is configured to adjust at least one of an angle, a direction, an intensity, and a distance of the beam of radiation.

20. The system of claim 18, wherein the means for controlling the computer controlled surgical instrument is configured to turn on and shut off the beam of radiation.

21. A method of surgically treating a tumor, comprising:
placing at least one datum device a predetemined distance from a targeted area, wherein a tumor is contained within the targeted area;
sensing the at least one datum device;
determining the position of the at least one datum device;
conveying information about the position of the at least one datum device to a surgical device for treating the tumor;
treating the tumor;
repeatedly calculating changes in the position of the at least one datum device;
conveying information about changes in the position of the at least one datum device to the surgical device; and
controlling the surgical device in response to the conveyed information.

22. The method of claim 21, wherein placing at least one datum device includes surgically implanting the at least one datum device.

23. The method of claim 21, wherein placing at least one datum device includes placing the at least one datum device on the patient's skin.

24. The method of claim 21, wherein determining the position of the at least one datum device includes triangulating signals from three datum devices.

25. The method of claim 21, wherein controlling the surgical device includes controlling a radiation beam emitted by the device.

26. The method of claim 25, wherein controlling the radiation beam includes adjusting at least one of an angle, a direction, an intensity, and a distance of the radiation beam.

27. The method of claim 21, wherein controlling the surgical device includes turning the device on and off.

28. The method of claim 25, wherein controlling the radiation beam includes turning the radiation beam on and off.

29. The method of claim 21, wherein sensing the at least one datum device includes receiving a signal transmitted from the at least one datum device.

30. The method of claim 21, wherein sensing the at least one datum device includes using a proximity sensor to locate the at least one datum device.

31. A system for surgically treating a tumor, comprising:
at least one datum device;
a surgical device for treating a tumor;
means for determining the position of the at least one datum device;
means for calculating changes in position of the at least one datum device in real-time;
means for conveying change in position information to the surgical device; and
means for controlling surgical device in response to the conveyed information.

32. The system of claim 31, further comprising three datum devices.

33. The system of claim 32, wherein the means for determining the position of the datum devices includes triangulation capabilities.

34. The system of claim 31, wherein the surgical device for treating a tumor is a radiotherapy device.

35. The system of claim 31, wherein the surgical device for treating a tumor is configured to emit a beam of radiation.

36. The system of claim 31, wherein the means for controlling the surgical device includes means for adjusting a variable output of the device.

37. The system of claim 35, wherein the means for controlling the surgical device includes means for adjusting at least one of an angle, a direction, an intensity, and a distance of the beam of radiation.

38. The system of claim 31, wherein the means for controlling surgical device includes means for turning on and shutting off device.

39. The system of claim 35, wherein the means for controlling the surgical device includes means for turning on and shutting off the beam of radiation.

40. A method of surgically treating a tumor, comprising:
applying a radiation beam to an area containing a tumor;
tracking changes in the position of the area in real-time; and
controlling the radiation beam in response to changes in the position of the area.

41. A system for surgically treating a tumor, comprising:
means for applying a radiation beam to an area containing a tumor;
means for tracking changes in the position of the area in real-time; and
means for controlling the radiation beam in response to changes in the position of the area.
